(19) United States
(12) Patent Application Publication
(10) Pub. No.: US 2026/0096923 A1
(43) Pub. Date: Apr. 9, 2026

(54) MULTI-CHANNEL AUTOMATIC INFUSION VALVE

(71) Applicant: Alcon Inc., Fribourg (CH)

(72) Inventors: Kevin CANZONE, Coto de Caza, CA (US); Jonathan MOREIRA, Rancho Margarita, CA (US); Behzad HASHEMI, Irvine, CA (US)

(21) Appl. No.: 19/351,080

(22) Filed: Oct. 6, 2025

Related U.S. Application Data
(60) Provisional application No. 63/705,127, filed on Oct. 9, 2024.

(51) Int. Cl. A61F 9/00; A61M 39/22; A61M 39/00

(52) U.S. Cl. CPC A61F 9/0017; A61M 39/223; A61M 39/227; A61M 2039/0009; A61M 2202/0007; A61M 2202/04; A61M 2205/3331; A61M 2205/7536; A61M 2206/10; A61M 2210/0612

(57) ABSTRACT

The present disclosure generally relates to fluid control valves for delivering and/or aspirating fluid during surgical surgeries and procedures. In one embodiment, an apparatus comprises a valve body comprising a first conduit defining a first port, and a second conduit defining a second port and a third port. A cavity is defined in the valve body between the first conduit and the second conduit. The cavity is fluidly coupled with the first conduit via a first channel having a smaller diameter, and with the second conduit via at least one second channel. The apparatus further comprises a hydrophobic filter attached to the valve body and extending substantially across the cavity. The apparatus further comprises an activation member disposed in the cavity and defining one or more openings extending from a first side of the activation member contacting the hydrophobic filter, to an opposing second side facing the second conduit.

### CROSS-REFERENCE TO RELATED APPLICATIONS

This application claims the benefit of U.S. Provisional Patent Application No. 63/705,127, filed October 9, 2024, which is incorporated by reference herein in its entirety, and is hereby expressly made a part of this specification.

### INTRODUCTION

Microsurgical procedures frequently require precision cutting, removal, and manipulation of various body tissues. For example, certain ophthalmic surgical procedures, such as pars plana vitrectomies, require cutting and removal of portions of the vitreous humor, a transparent gel-like material that fills the posterior segment of the eye. Simultaneously, while removing the vitreous humor, a liquid solution (e.g., balanced salt solution (BSS)) is typically infused into the eye to maintain intraocular pressure and prevent collapse of the eye wall. In cases of retinal breaks or retinal detachment, the liquid solution may then be exchanged for air (or other gas(es)), through a process known as fluid-air exchange, to help push out subretinal fluid from the intraocular space while maintaining intraocular pressure and temporarily holding the retina in place. During such procedures, the liquid solution and air are provided by separate supply lines that are conjoined with a singular downstream infusion line via a stopcock.

In some cases, the air pressure in the gas supply line may build up and cause air to escape into the infusion line, forming air bubbles in the infusion liquid which may travel to the eye and negatively affect intraocular pressure during surgery. Existing designs for check valves of infusion stopcocks, however, do not allow venting through the gas supply line without reverse leakage of liquid and thus, there is currently no effective way to remove the air bubbles or prevent their escape into infusion liquids. Additionally, during some procedures, infusion fluids must be back-flowed through the infusion line in order for other surgical fluids, such as a retinal tamponade, to be injected into the intraocular space. In such cases, the amount of infusion fluid that may be back-flowed is limited by the inability to purge infusion gases through the gas supply line without reverse leakage of liquids therein, which may damage the air pump and/or cause additional complications during fluid infusion.

Therefore, what is needed in the art are improved fluid control valves for ophthalmic fluid infusion that enable aspiration and purging of gases.

### SUMMARY

Aspects of the present disclosure generally relate to devices for surgical procedures (e.g., to surgical devices for ophthalmic intraocular fluid delivery (e.g., infusion) and aspiration).

Certain embodiments of the present disclosure provide an apparatus to perform fluid infusion during surgical procedures. The apparatus comprises a valve body comprising a first conduit defining a first port, and a second conduit defining a second port and a third port. A cavity is defined in the valve body between the first conduit and the second conduit. The cavity is fluidly coupled with the first conduit via a first channel having a diameter smaller than that of the first conduit, and with the second conduit via at least one second channel. The apparatus further comprises a hydrophobic filter attached to the valve body and extending substantially across the cavity. The apparatus further comprises an activation member disposed in the cavity and defining one or more openings extending from a first side of the activation member contacting the hydrophobic filter, to an opposing second side facing the second conduit.

The following description and the related drawings set forth herein detail certain illustrative features of one or more embodiments, including those described above.

### BRIEF DESCRIPTION OF THE DRAWINGS

The drawings described herein are for illustrative purposes only, are schematic in nature, and are intended to be exemplary rather than to limit the scope of the disclosure.

FIG. 1 illustrates a perspective view of an exemplary surgical console, according to embodiments described herein.

FIG. 2A illustrates a perspective view of an exemplary valve assembly, according to embodiments described herein.

FIG. 2B illustrates a perspective exploded view of the valve assembly of FIG. 2A, according to embodiments described herein.

FIG. 2C illustrates a schematic cross-sectional view of the valve assembly of FIG. 2A, according to embodiments described herein.

FIG. 2D illustrates an enlarged view of a portion of FIG. 2C including a cavity formed by the valve assembly, according to embodiments described herein.

FIG. 2E illustrates a perspective top view of the base of the bottom portion of the valve assembly of FIG. 2A, according to embodiments described herein.

FIG. 2F illustrates a perspective bottom view of the cover of the top portion of the valve assembly of FIG. 2A, according to embodiments described herein.

FIG. 3A illustrates a schematic plan view of an exemplary operational mode of the valve assembly of FIGS. 2A-2F, according to embodiments described herein.

FIG. 3B illustrates a schematic plan view of an exemplary operational mode of the valve assembly of FIGS. 2A-2F, according to embodiments described herein.

FIG. 3C illustrates a schematic plan view of an exemplary operational mode of the valve assembly of FIGS. 2A-2F, according to embodiments described herein.

The above summary is not intended to represent every possible embodiment or every aspect of the subject disclosure. Rather the foregoing summary is intended to exemplify some of the novel aspects and features disclosed herein. The above features and advantages, and other features and advantages of the subject disclosure, will be readily apparent from the following detailed description of representative embodiments and modes for carrying out the subject disclosure when taken in connection with the accompanying drawings and the appended claims.

### DETAILED DESCRIPTION

The present disclosure generally relates to fluid control valves for delivering and/or aspirating fluid during surgical procedures (e.g., ophthalmic procedures). For example, the fluid control valves described herein may be used during vitreoretinal procedures, such as pars plana vitrectomies for the treatment of posterior segment diseases. Vitrectomies typically require cutting and removal of portions of the vitreous humor. In order to maintain intraocular pressure and prevent collapse of the eye during such surgical procedures, liquid is infused into the intraocular space and thereafter aspirated. In certain procedures, the liquid is then exchanged with air or other gases during a process known as fluid-air exchange. During such processes, it is typically beneficial to purge or vent any undesired gases in the infusion line and/or the intraocular space to maintain intraocular pressure. The fluid control valves and methods described herein provide improved structures and mechanisms for infusion fluid flow regulation that enable upstream purging and/or venting of gases from infusion lines while also preventing liquids from the infusion lines to leak into the gas supply lines.

In the following description, details are set forth by way of example to facilitate an understanding of the disclosed subject matter. It should be apparent to a person of ordinary skill in the field, however, that the disclosed implementations are exemplary and not exhaustive of all possible implementations. Thus, it should be understood that reference to the described examples is not intended to limit the scope of the disclosure. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one implementation may be combined with the features, components, and/or steps described with respect to other implementations of the present disclosure. Further, while many of the examples provided herein relate to ophthalmic surgical procedures, other surgical procedures types are also contemplated.

Note that, as described herein, a distal end, segment, or portion of a component refers to the end, segment, or portion that is closer to a patient’s body during use thereof. On the other hand, a proximal end, segment, or portion of the component refers to the end, segment, or portion that is distanced further away from the patient’s body and is in proximity to, for example, a surgical console.

In certain embodiments, an apparatus to perform fluid infusion during ophthalmic procedures is described. The apparatus comprises a valve body comprising a first conduit defining a first port, and a second conduit defining a second port and a third port. A cavity is defined in the valve body between the first conduit and the second conduit. The cavity is fluidly coupled with the first conduit via a first channel having a diameter smaller than that of the first conduit, and with the second conduit via at least one second channel. The apparatus further comprises a hydrophobic filter attached to the valve body and extending substantially across the cavity. The apparatus further comprises an activation member disposed in the cavity and defining one or more openings extending from a first side of the activation member contacting the hydrophobic filter, to an opposing second side facing the second conduit.

In some embodiments, the hydrophobic filter is configured to prevent the backflow of liquid into an air supply line. In some embodiments, the activation member is ring-shaped and is formed of a hydrophobic material and/or of a material having a hardness equal to or less than that of the hydrophobic filter. The equal-or-lesser hardness of the material of the activation member helps avoid damage to the hydrophobic filter that may occur during contact. Any damage that is sustained by the hydrophobic filter tends to reduce its performance or render it non-functional.

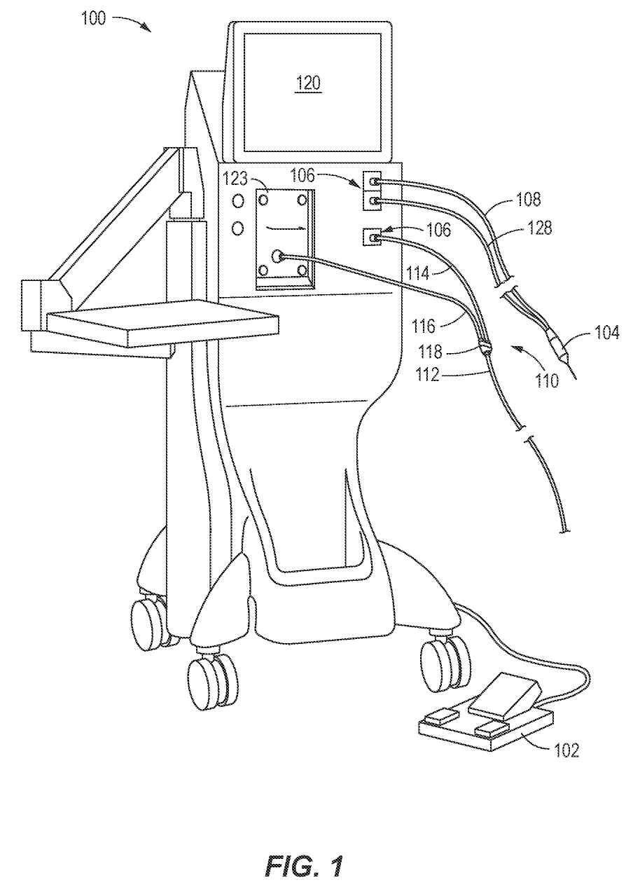

FIG. 1 illustrates a perspective view of an exemplary surgical console 100 that may be utilized in combination with the fluid control valves described herein. The surgical console 100 is operably coupled, physically or wirelessly, to any number of user interfaces, including a foot controller 102 and a surgical tool 104, such as a vitrector. The surgical console 100 provides one or more port connectors 106 for physically coupling the user interfaces to various components or subcomponents of the surgical console 100. For example, the surgical tool 104 may be fluidly coupled with a pneumatic source of the surgical console 100, via a pneumatic line 128 connected to a port connector 106, to facilitate a reciprocating motion of a cutter of the surgical tool 104 for cutting vitreous in a patient’s eye. Further, the surgical tool 104 may be fluidly coupled with a vacuum source, via a vacuum supply line 108 connected to a port connector 106, to enable aspiration of cut vitreous from the patient’s eye.

Similarly, one or more port connectors 106 may be utilized to couple a fluid infusion system 110 with one or more infusion fluid sources, (e.g., an air/gas source, a liquid perfluorocarbon source, a silicone oil infusion (SOI) source, a BSS source, etc.) to enable infusion of fluids into the eye during vitreous removal. In certain embodiments, a liquid supply line 116 of the fluid infusion system 110 may be coupled to one or more infusion fluid sources via a fluidics subsystem 123. As shown in FIG. 1, the fluid infusion system 110 includes an infusion line 112 fluidly coupled with a gas supply line 114 and a separate liquid supply line 116 at a three-way automatic valve assembly 118, which may enable selective flow of different infusion fluids through the infusion line 112.

In operation, the user may control an aspect or mechanism of the surgical tool 104 and/or the fluid infusion system 110 via actuation of the foot controller 102, which may include a foot pedal, or other user input device. For example, the user may press down on (e.g., depress) the foot controller 102 to initiate and/or increase a flow rate of an infusion fluid from a fluid source through the fluid infusion system 110 and into the eye of the patient. Alternatively, reducing the depression of the foot controller 102 (e.g., lifting the user’s foot) may decrease and/or ultimately stop the flow of fluid through the fluid infusion system 110. Accordingly, in certain embodiments, the flow rate of infusion fluids through the fluid infusion system 110 corresponds to the amount of depression of the foot controller 102.

In certain embodiments, the surgical console 100 further includes a display 120 for displaying information to the user. Thus, the display 120 may display information about infusion fluid parameters, such as infusion fluid flow rates and intraocular pressure, to the user during operation thereof, as well as information related to the performance of the surgical tool 104. In some cases, the display 120 may also incorporate an input device (e.g., a touchscreen overlaid or integrated with display hardware) for receiving user input.

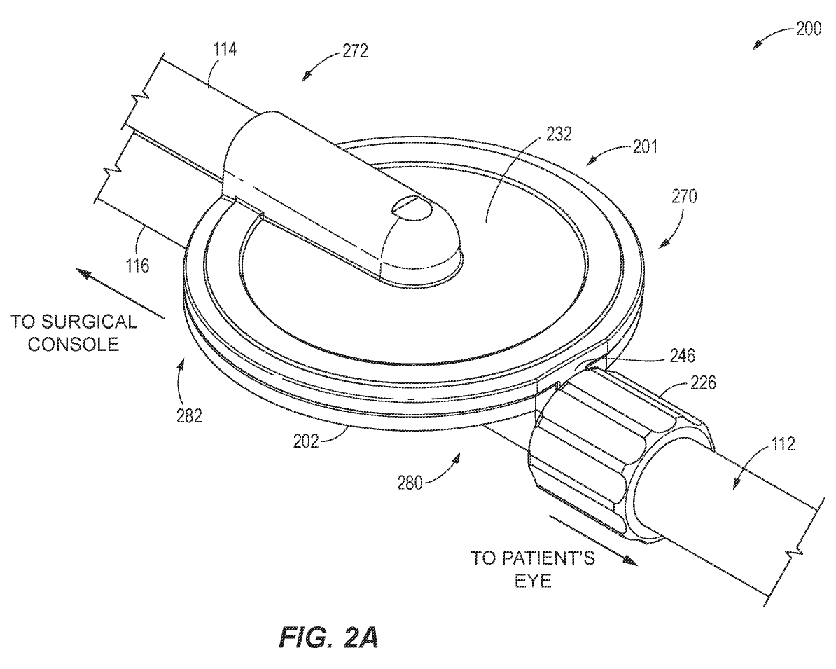

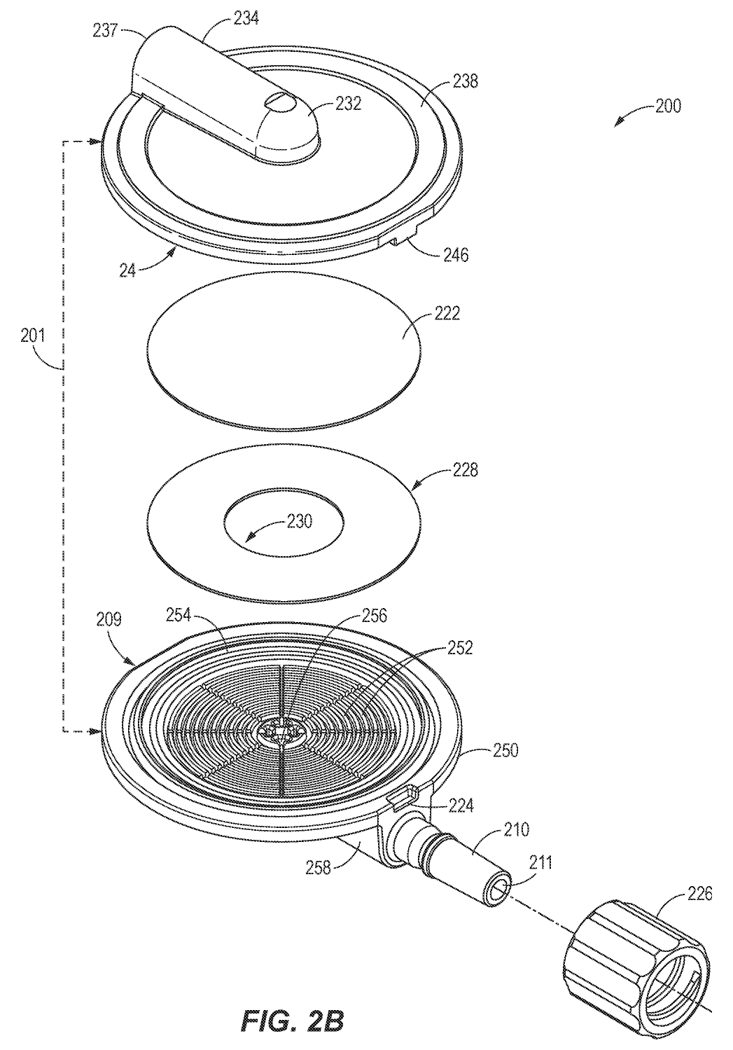

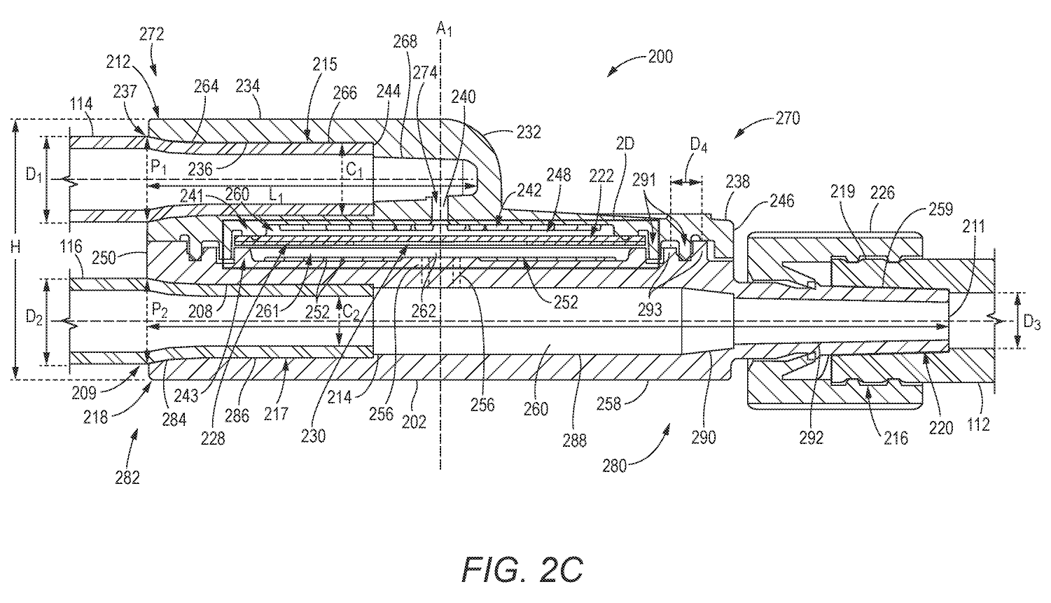

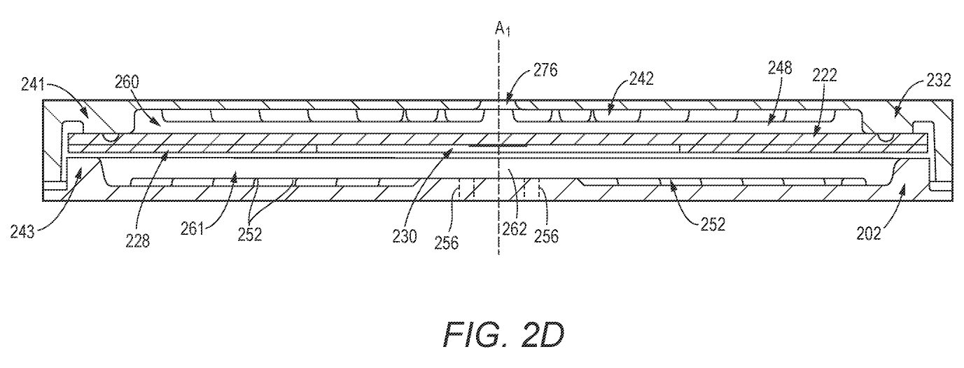

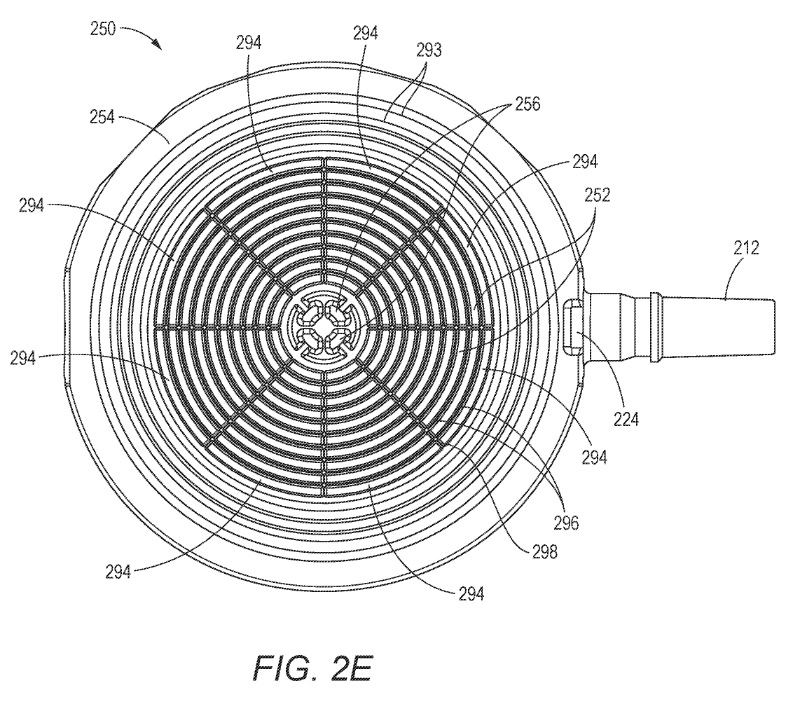

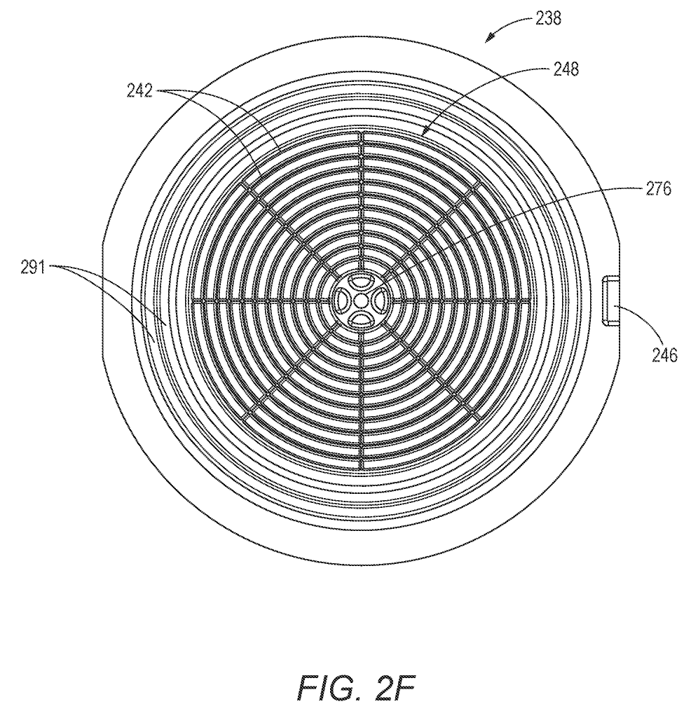

FIGS. 2A-2F illustrate a valve assembly 200 for flow control of infusion fluids during surgical procedures, according to one or more embodiments. More specifically, FIG. 2A illustrates a perspective view of the valve assembly 200; FIG. 2B illustrates a perspective exploded view of the valve assembly 200; FIG. 2C illustrates a cross-sectional view of the valve assembly 200; FIG. 2D illustrates an enlarged view of a portion of FIG. 2C including a cavity formed by the valve assembly 200; FIG. 2E illustrates a perspective top view of the second portion (e.g., lower body) of the valve assembly 200; and FIG. 2F illustrates a perspective bottom view of the first portion (e.g., upper body) of the valve assembly 200. FIGS. 2A-2F are described together herein for clarity.

The valve assembly 200 represents one example implementation of the three-way automatic valve assembly 118, which may be utilized in combination with the fluid infusion system 110, surgical tool 104, and/or the surgical console 100 described above. The valve assembly 200 generally includes a hydrophobic filter 222 (also referred to as “filter 222”) and an activation member 228 that are disposed within a cavity 248 formed within a valve body 201. In some embodiments, the valve body 201 comprises a first portion (e.g., an upper body 232 or “cover”) that fluidly couples with the gas supply line 114, and a second portion (e.g., a lower body 202 or “base”) that fluidly couples with a liquid supply line 116. In some embodiments, the cavity 248 is defined when the first portion and the second portion are coupled together (e.g., when the valve assembly 200 is in an assembled state). The dimensioning and positioning of the hydrophobic filter 222 are controlled such that the hydrophobic filter 222 extends substantially across the cavity, which allows bidirectional gas flow (e.g., air) through the hydrophobic filter 222 between the gas supply line 114 and the infusion line 112, and prevents liquid flow through the hydrophobic filter 222 into or toward the gas supply line 114. Thus, gases may be vented or purged from the fluid infusion system 110 during fluid infusion to enable improved control of intraocular pressure during surgical procedures.

As noted above, the valve body 210 generally includes an upper body 232 configured to interface (e.g., couple) with a lower body 202. In certain embodiments, the upper body 232 and/or lower body 202 are formed of any suitable plastic or thermoplastic materials, such as acrylonitrile butadiene styrene (ABS), polycarbonate (PC), nylon, and acrylic, which may be transparent or opaque in color. In certain embodiments, the upper body 232 and lower body 202 are formed of the same material, while in other embodiments, upper body 232 and lower body 202 are formed of different materials.

In certain embodiments, a distal end 270 of the upper body 232 may include a key 246 that mates with a cutout 224 formed at a distal end 280 of the lower body 202 to ensure that the upper body 232 and lower body 202 are correctly (e.g., rotationally and laterally) aligned when the valve assembly 200 is assembled. Although a single key 246 and cutout 224 are shown, additional keys and/or cutouts may also be formed in the upper body 232 and/or lower body 202 to facilitate alignment between upper body 232 and lower body 202, such as along perimeters thereof. Further, the disposition of the key 246 and cutout 224 may be switched between the upper body 232 and the lower body 202, such that the upper body 232 includes the cutout 224 and the lower body includes the key 246.

In some embodiments, when the valve assembly 200 is in an assembled state, the upper body 232 and the lower body 202 are permanently or semi-permanently attached together using any suitable techniques. In one example implementation, the upper body 232 and the lower body 202 are welded together, such as by ultrasonic welding. Other example implementations include applying an adhesive material, fasteners, and so forth between the upper body 232 and lower body 202.

Turning to FIG. 2C, in certain embodiments, the upper body 232 and the lower body 202 are configured such that, when assembled, the valve assembly 200 has a height H that is not substantially greater than the combined outer diameters (or widths) D₁ and D₂ of the liquid supply line 116 and the gas supply line 114, respectively. For example, the height H of the valve assembly 200 may be less than 150%, 140%, 130%, 120%, or 110% or less, than a combined outer diameter or width (e.g., D₁ + D₂) of the liquid supply line 116 and the gas supply line 114.

The upper body 232 includes a cover 238 from which an arm 234 extends in a proximal direction (e.g., toward a surgical console or gas source, and toward the proximal end 272 of the upper body 232) for coupling with gas supply line 114. The gas supply line 114 couples with a port 237 at a proximal end 212 of the arm 234, which provides fluid connection with a conduit 236 extending through a length L₁ of the arm 234. In certain embodiments, the conduit 236 comprises a first tapered portion 264 at a proximal end of the conduit 236 and having a non-uniform diameter (or width), a second tapered portion 268 at a distal end of the conduit 236 and having a non-uniform diameter (or width), and a uniform portion 266 disposed between the first tapered portion 264 and the second tapered portion 268 and having a uniform diameter (or width).

In certain embodiments, a diameter (or width) P₁ of the port 237 at the proximal end 212 of the arm 234 is slightly smaller, substantially the same, or slightly larger than the outer diameter D₁ of the gas supply line 114 to allow a distal end 215 of the gas supply line 114 to be securely fit within the port 237. In certain embodiments, the diameter P₁ of the port 237 is greater than a diameter C₁ of the uniform portion 266 of the conduit 236, such that the first tapered portion 264 forms a sloped, decreasing transition in diameter between the port 237 and the uniform portion 266. Accordingly, the diameter of the first tapered portion 264 decreases in a distal direction from the proximal end 212 of the arm 234. Similarly, in certain embodiments, the diameter of the second tapered portion 268 decreases in a distal direction from the proximal end 212 of the arm 234.

In certain embodiments, the arm 234 includes a step 244 formed between the uniform portion 266 and the second tapered portion 268 of the conduit 236. As a result of the step 244, there is a sudden and abrupt decrease in diameter between a distal end of the uniform portion 266 and a proximal end of the second tapered portion 268. In certain embodiments, the step 244 provides a manual stop for the gas supply line 114, which enables the user (e.g., a surgeon) to consistently insert the gas supply line 114 into the conduit 236 along a predetermined distance for each procedure.

A proximal end 274 of a channel 240 fluidly couples with the second tapered portion 268 of the conduit 236. While the channel 240 extends from the second tapered portion 268, the channel 240 does not, in certain embodiments, extend from a most distal end of the second tapered portion 268; rather, the channel 240 extends from a position between a most proximal end and the most distal end of the second tapered portion 268. As shown, the most distal end of the second tapered portion 268 may have a slightly rounded, or dome-shaped morphology distally beyond the channel 240.

The channel 240 extends from the second tapered portion 268 along a first axis A1 that is non-parallel to a second axis A2 of the conduit 236. In certain embodiments, and as shown in FIG. 2C, the first axis A1 is normal to, or disposed at an angle of 90° relative to, the second axis A2. In other embodiments, the first axis A1 is disposed at an angle greater than or less than 90° relative to the second axis A2.

In certain embodiments, the channel 240 has a diameter (or width) less than any diameter or width of the port 237 and the conduit 236. In certain embodiments, the channel 240 has a uniform, or consistent, diameter from the proximal end 274 of the channel 240 nearest the second tapered portion 268 to a distal end 276 of the channel 240 furthest from the second tapered portion 268. In certain embodiments, the channel 240 has a non-uniform diameter from the proximal end 274 to the distal end 276. For example, the channel 240 may comprise one or more steps or sloped transitions in diameter between the proximal end 274 and the distal end 276. As shown in FIG. 2C, in certain embodiments, a diameter of the channel 240 at the distal end 276 is greater than a diameter of the channel 240 at the proximal end 274.

As mentioned above, the cavity 248 may be defined when the upper body 232 and the lower body 202 of the valve body 201 are coupled together. As shown, the cover 238 has a disc-like shape and further defines one or more ridges (e.g., ribs or grooves) 242 extending from a lower surface of the cover 238 that define one or more channels within the cavity 248. In certain embodiments, the ridges 242 are annular or semi-annular ridges that circumscribe the distal end 276 of the channel 240. In some cases, the ridges 242 provide added mechanical support for the filter 222 when the valve assembly 200 is in an assembled state.

In certain embodiments, the decreased diameter of the channel 240 relative to the conduit 236 may be configured to increase the localized velocity of gas supplied from the gas supply line 114 as the gas enters the cavity 248 and contacts the filter 222. The additional velocity created by the decreased diameter of the channel 240 may provide sufficient force on the filter 222 to create a “diaphragm” effect on the filter 222 during use, as discussed elsewhere herein.

The lower body 202 of the valve assembly 200 includes a base 250 that is coupled to a flow-through member 258. In some embodiments, the base 250 and the flow-through member 258 are integrally formed. The flow-through member 258 comprises a port 209 at a proximal end 218 of the flow-through member 258 for coupling with the liquid supply line 116, and which further provides fluid connection with a second conduit 208 extending through a length L₂ of the flow-through member 258 between the proximal end 218 and a distal end 220 of the flow-through member 258.

In certain embodiments, the second conduit 208 comprises a first tapered portion 284 at a proximal end of the second conduit 208 and having a non-uniform diameter (or width), a second tapered portion 290 at a distal end of the second conduit 208 and having a non-uniform diameter (or width), a third tapered portion 292 at a distal end of the second conduit 208 and having a non-uniform diameter (or width), a first uniform portion 286 disposed distal to the first tapered portion 284 and having a uniform diameter (or width), and a second uniform portion 288 disposed between the first uniform portion 286 and the second tapered portion 290 and having a uniform diameter (or width).

In certain embodiments, a diameter P₂ of the port 209 is slightly smaller, substantially the same, or slightly larger than the outer diameter D₂ of the liquid supply line 116 to allow a distal end 217 of the liquid supply line 116 to be securely fit within the port 209. In certain embodiments, the diameter P₂ of the port 209 is greater than a diameter C₂ of the first uniform portion 286 of the second conduit 208, such that the first tapered portion 284 forms a sloped, decreasing transition in diameter between the port 209 and the first uniform portion 286. Accordingly, the diameter of the first tapered portion 284 decreases in a distal direction from the proximal end 218 of the flow-through member 258. Similarly, in certain embodiments, the diameters of the second tapered portion 290 and the third tapered portion 292 decrease in a distal direction from the proximal end 218 of the flow-through member 258.

In certain embodiments, the proximal end 218 of the flow-through member 258 may include a step 214 formed between the first uniform portion 286 and the second uniform portion 288. As a result of the step 214, there is a sudden and abrupt decrease in diameter between a distal end of the first uniform portion 286 and a proximal end of the second uniform portion 288. In certain embodiments, the step 214 provides a manual stop for the liquid supply line 116, which enables the user (e.g., a surgeon) to consistently insert the liquid supply line 116 into the second conduit 208 along a predetermined distance for each procedure. Further, the step 214, in addition to the second tapered portion 290 and the third tapered portion 292, may be configured to improve fluidics through the lower body 202 and/or to ensure the liquid entering the eye is free of unwanted air bubbles.

The distal end 220 of the flow-through member 258 generally sized to have an outer diameter slightly smaller than an inner diameter of the proximal end of the infusion line 112, and that includes the third tapered portion 292 of the second conduit 208 formed therethrough. Accordingly, the infusion line 112 is configured to securely fit around the mating feature 259. In certain embodiments, the distal end of the flow-through member 258 may further include a locking mechanism disposed around the mating feature 259, such as a Luer lock 226, that is configured to provide additional mechanical holding force to secure the infusion line 112 to the valve assembly 200 and create a leak-free seal between the mating feature 259 and the infusion line 112. For example, the Luer lock 226 may comprise a threaded interior surface 216 through which the proximal end 219 of the infusion line 112 may be secured within.

The second conduit 208 (e.g., the flow-through member 258) extends along a third axis A3 that is non-parallel to the first axis A1 of the channel 240. In certain embodiments, the third axis A3 is normal to, or disposed at an angle of 90° relative to, the first axis A1. In other embodiments, the third axis A3 is disposed at an angle greater than or less than 90° relative to the first axis A1. In certain embodiments, the third axis A3 of the second conduit 208 is parallel to the second axis A2 of the conduit 236 (e.g., where the second axis A2 and the third axis A3 are each disposed at 90° relative to the first axis A1).

The base 250 of the lower body 202 is configured to interface and engage with the cover 238 of the upper body 232 and retain the filter 222 and the activation member 228 therebetween. In some embodiments, when the valve assembly 200 is in an assembled state, the filter 222 extends substantially across the cavity 248. As used herein, “substantially across” indicates that the configuration of the filter 222 (e.g., the dimensioning and the positioning within the cavity 248) is effective to prevent liquid flow across the filter 222 (e.g., the filter 222 extends across the flow path). In some embodiments, the filter 222 partitions the cavity 248, and defines a first (non-liquid-side) cavity 260 and a second (liquid-side) cavity 261.

In some embodiments, the lower surface of the cover 238 defines raised features 241 that are arranged radially (or laterally) outward of the one or more ridges 242. In some embodiments, the raised features 241 are defined by one or more annular or semi-annular ridges formed in the cover 238. In some embodiments, the raised features 241 have a greater height than the one or more ridges 242, e.g., along the first axis A1. The filter 222 contacts the raised features 241, and in some embodiments the filter 222 is attached to the cover 238 at the raised features 241 using any suitable techniques, such as by welding via ultrasonic welding, or the like. In alternate embodiments, the filter 222 may be attached to other portions of the cover 238 and/or the base 250 to extend substantially across the cavity 248. In some embodiments, the filter may be welded (e.g., ultrasonically welded) to the cover 238 and then the cover 238 may be welded (e.g., ultrasonically welded) to the lower body 202 / base member. Other methods of attachment are also contemplated (e.g., adhesive).

The activation member 228 contacts the filter 222 and defines one or more openings through the activation member 228. In some embodiments, the activation member 228 is attached to the filter 222 or to other portions of the cover 238 and/or base 250 to provide the contact between the activation member 228 and the filter 222. In the illustrated embodiments, the activation member 228 is a ring-shaped membrane or layer and defines a central opening 230 that is circle-shaped. The outer diameter of the activation member 228 is shown as the same as the diameter of the filter 222. However, other shapes, numbers, and arrangements of openings (e.g., two or more openings), as well as different dimensioning of the activation member 228, are also contemplated. Further discussion of the activation member 228 is provided below.

In some embodiments, the base 250 defines raised features 243 that are arranged radially (or laterally) outward of the one or more ridges 252. In some embodiments, the raised features 243 are defined by one or more annular or semi-annular ridges formed in the base 250. In some embodiments, the raised features 243 have a greater height than the one or more ridges 252, e.g., along the first axis A1. The raised features 243 may extend outward of the raised features 241, e.g., where the raised features 241, 243 are partly overlapping or non-overlapping with each other.

In some embodiments, the filter 222 and/or the activation member 228 contacts the raised features 243 when the valve assembly 200 is in an assembled state (e.g., such that the filter 222 and/or the activation member 228 in a neutral configuration is “sandwiched” between the raised features 241, 243). In other embodiments, the filter 222 and/or the activation member 228 need not contact the raised features 243 while extending substantially across the cavity 248.

In certain embodiments, the base 250 has one or more mating features 293 formed on an upper surface thereof that are configured to mate, or engage with, corresponding mating features 291 formed on a lower surface of the cover 238 when the valve assembly 200 is assembled. Such mating features 293 and 291, in addition to the key 246 and cutout 224, may facilitate lateral and/or rotational alignment of the lower body 202 with the upper body 232. In certain embodiments, the mating features 293 and 291 are disposed radially outward of the cavity 248 and radially inward of an outer circumference of the base 250 and the cover 238, respectively. In certain embodiments, as best seen in FIGS. 2E-2F, the mating features 293 and 291 are annular, and follow along an entire outer circumference of the base 250 and cover 238, respectively. In certain embodiments, each of the mating features 293 and 291 are semi-annular and follow along only a portion of the outer circumference (e.g., along 25% of the circumference, or along 50% of the circumference) of the base 250 and cover 238, respectively. In such embodiments, the mating features 293 and 291 may each comprise a plurality of features arranged in a circumferential manner and separated by one or more gaps. Generally, the mating features 293 may comprise grooves, protrusions, and/or other similar features, while mating features 291 may comprise corresponding grooves, protrusions, and/or other similar features configured to mate with mating features 293.

In some embodiments, the one or more mating features 291, 293 each comprise a plurality of mating features that are spaced apart from each other. For example, the mating features 293 are shown as being spaced apart by a pitch distance D4, which may depend on the material composition of the base 250 and the cover 238. In some embodiments, the pitch distance D4 ranges between about 500 µm (micrometer) to about 1 mm (millimeter).

Similar to the cover 238, the base 250 includes one or more ridges 252 extending from an upper surface of the base 250 to partly define the cavity 248. In some embodiments, the ridges 252 are configured to provide mechanical support to the filter 222 and/or the activation member 228 when the valve assembly 200 is in an assembled state. The ridges 252 may be densely populated within the base 250, such that there is very little volume within the base 250 not occupied by ridges 252. The dense positioning of the ridges 252 within the base 250 may limit the volume available within the cavity 248 for fluid to enter during fluid-air exchange operations.

Generally, the ridges 252 may have any suitable shape and/or arrangement. In certain embodiments, as shown in FIG. 2E, the ridges 252 are semi-annular and are disposed in concentric rings 294 that increase in size (e.g., diameter/circumference) with increased distance of the concentric ring 294 from a center 262 of the base 250. The concentric rings (e.g., see concentric ring 294 in FIG. 2E) of ridges 252 form corresponding concentric rings of channels 296 between adjacent rings of ridges 252.

Further, in embodiments with semi-annular ridges 252 arranged in concentric rings 294, the semi-annular ridges 252 in each concentric ring 294 may increase in length with increased distance of the concentric ring 294 from the center 262. In certain embodiments, each concentric ring 294 comprises breaks or gaps 298 between the ridges 252 thereof, and in certain embodiments, the gaps 298 of some or all of the concentric rings 294 may be circumferentially aligned. As shown in FIG. 2E, alignment of the gaps 298 of all the concentric rings 294 in base 250 may form linear channels extending radially outward from the center 262.

Turning to FIG. 2F, the cavity 248 of the upper body 232 may also include semi-annular ridges 242 as discussed herein with reference to FIG. 2E. The semi-annular ridges 242 may be arranged in concentric rings creating corresponding annular channels between each ring of ridges 242, and gaps between the ridges 242 may form straight channels through the concentric rings that extend outward from a center of the cavity 248. Though a specific arrangement of ridges and channels is described and shown herein, alternate arrangements of ridges and channels in the cavity 248 are also contemplated.

Returning to FIG. 2C, when the valve assembly 200 is in an assembled state, the filter 222 partitions the cavity 248, defines the first cavity 260 between a bottom surface of the cover 238 and a top surface of the filter 222, and defines the second cavity 261 between a bottom surface of the filter 222 and a top surface of the base 250. In some embodiments, the first cavity 260 and the second cavity 261 provide clearance spaces that allow the filter 222 and/or the activation member 228 to deform toward the cover 238 and/or toward the base 250 responsive to gas flow and/or liquid flow causing pressure changes, e.g., during fluid-air exchange operations. In certain embodiments, the height of the second cavity 261 is optimized to allow a controlled fluid volume to enter the base 250 and occupy the second cavity 261. The clearance space may be further configured to allow for movement of the filter 222 to contact the ridges 242 and/or the ridges 252 during fluid-air exchange operations as further described below.

In certain embodiments, the ridges 252 circumscribe proximal ends of one or more second channels 256 (also referred to as “through channels” or “ports”) that fluidly couple the cavity 248 with the second conduit 208 of the flow-through member 258. Accordingly, the port 237 at the proximal end 272 of the upper body 232 is fluidically coupled to the port 211 at the distal end 280 of the lower body 202 via the combination of the first conduit 236, the channel 240, the cavity 248, the through channels 256, and the second conduit 208.

In some embodiments, the first channel 240, the cavity 248, and the one or more second channels 256 are coaxially disposed about the first axis A1. In some embodiments, the through channels 256 include two or more channels disposed through a center 262 of the base 250 and fluidly coupling the cavity 254 with the second conduit 208. A proximal end of each through channel 256 is disposed adjacent to the base 250, while a distal end of each through channel 256 is disposed adjacent to the second conduit 208.

In some embodiments, the one or more second channels 256 comprises two or more second channels that are disposed circumferentially about the first axis. For example, the through channels 256 may include two, three, four, five, or more through channels 256 arranged in a circular arrangement through the base 250 (four through channels 256 are shown in FIG. 2D). In certain embodiments, the through channels 256 may each have a curved and/or oblong shape, as pictured in FIG. 2D. In certain other embodiments, the through channels 256 may be a circular, oval, or polygonal shape. In certain embodiments, each through channel 256 is uniform in lateral dimensions and/or shape through the base 250 (e.g., the proximal and distal ends of each through channel 256 may have the same dimensions). In certain embodiments, each through channel 256 is non-uniform in lateral dimensions and/or shape through the base 250 (e.g., the proximal and distal ends of each through channel 256 may have different dimensions). In certain embodiments, one through channel 256 of the plurality of through channels 256 may be different in dimensions and/or shape from another through channel 256 of the plurality of through channels 256.

The filter 222, disposed in the cavity 248 between the upper body 232 and lower body 202 may include any suitable type of membrane filter having a hydrophobic membrane that is permeable to gas. The hydrophobic membrane may also be capable of capturing individual viruses and bacteria, thus acting as a sterile barrier (e.g., filter) to prevent viruses and bacteria from entering the eye from the low-pressure gas force.

In some examples, the filter 222 includes a hydrophobic membrane formed of polytetrafluoroethylene (PTFE), expanded PTFE (ePTFE), polycarbonate track etch (PCTE), polyesters (e.g., polyethylene terephthalate (PET)), radiation stable polyvinylidene difluoride (PVDF), nylon, cellulose (e.g., surfactant free cellulose acetate (SCFA), cellulose nitrate (CN), cellulose acetate (CA), polyethersulfone (PES), glass fibers, or acrylic copolymers. The hydrophobic membrane may further be unsupported or supported by a backing formed of materials including but not limited to polyester, polyethylene, polypropylene, or nylon. For example, in certain embodiments, the filter 222 includes an ePTFE membrane having a polyester backing. The hydrophobic membrane has a pore size ranging between about 0.1 µm (micrometer) to about 10.0 µm, such as between about 0.2 µm to about 5 µm, such as between about 0.5 µm to about 3.0 µm, such as between about 0.8 µm to about 1.2 µm. Furthermore, the hydrophobic membrane may have a thickness ranging between about 150 µm to about 400 µm, such as between about 200 µm to about 350 µm.

Please note that although a single filter 222 is depicted in FIGS. 2A-2D, it is further contemplated that the valve assembly 200 may include two or more filters arranged in a linear or stacked configuration. The two or more filters may be formed of different materials and/or have different pore sizes relative to each other. For example, in certain embodiments, a second filter having a finer pore size may be disposed upstream of the filter 222 to provide additional filtration of gases flowed through the gas supply line 114, while the filter 222 provides a hydrophobic barrier and prevent liquids from flowing therein.

The activation member 228 may be formed of any suitable materials that provide a low surface tension interface with the filter 222. In some embodiments, the material(s) forming the activation member 228 are gas-permeable. In some embodiments, the material(s) forming the activation member 228 are hydrophobic. In some embodiments, the upper surface of the activation member 228 comprises a hydrophobic surface that contacts the hydrophobic membrane of the filter 222. In some embodiments, the lower surface of the activation member 228 also comprises a hydrophobic surface. In other embodiments, the lower surface of the activation member 228 comprises a hydrophilic surface. In some embodiments, the activation member 228 is implemented as a filter, e.g., having a material composition similar to that of the filter 222 (e.g., as an ePTFE membrane having a polyester backing). In other embodiments, the material composition of the activation member 228 may be different from the filter 222, such as a different composition of the example materials discussed above that are used for the hydrophobic membrane and/or backing, or different material(s) altogether. For example, in some embodiments, the activation member 228 may include Ultra High Molecular Weight Polyethylene (UHMWPE) as the hydrophobic membrane or as the entire activation member 228. In some embodiments, the activation member 228 may be made of silicone or polydimethylsiloxane (PDMS). For example, a silicone/PDMS activation member 228 may be used with a radiation stable PVDF membrane.

A first side (e.g., the hydrophobic surface) of the activation member 228 contacts the filter 222 (e.g., the hydrophobic membrane), and an opposing second side of the activation member 228 faces the second conduit 208. In some embodiments, only a portion of the surfaces facing each other (e.g., the hydrophobic surface of the activation member 228 and the hydrophobic membrane of the filter 222) are contacting when in a neutral configuration. For example, the filter 222 and the activation member 228 may be attached to each other (and to the raised features 241 of the cover 238) near their respective circumferential (or lateral) edges, and the surfaces may contact each other near those edges. Away from the edges, the medial (or central) portions of the filter 222 and the activation member 228 may or may not contact each other. For example, the medial portions of the filter 222 and the activation member 228 may be permitted to move independently of each other responsive to gas flow and liquid flow. Generally, a smaller distance between the filter 222 and the activation member 228 corresponds to less pressure that is required to deform the filter 222 to contact the activation member 228 and thereby cause a squeezing effect, which is discussed below. In some embodiments, the activation member 228 may be non-fixed relative to the filter 222 such that that activation member 228 may be able to separate from the filter 222 (e.g., the filter 222 may be fixed relative to valve assembly and the activation member 228 may release from the filter 222 when air passes through the filter 222 toward the activation member 228.

During operation of the valve assembly 200, infusion liquid from the liquid source may flow through the liquid supply line 116, into the flow-through member 258 of the lower body 202, and through the infusion line 112 toward the patient’s eye and vice versa. Alternatively, infusion gases from the gas source may flow through the gas supply line 114, into the upper body 232, past the filter 222 and the activation member 228 into the lower body 202, and through the infusion line 112 toward the patient’s eye and vice-versa. The disposition of the hydrophobic filter 222 and the activation member 228 between the upper body 232 and the lower body 202 passively prevents the flow of liquids into the upper body 232 and the gas supply line 114, while allowing gases to pass therethrough. Thus, the valve assembly 200 facilitates venting, purging, and/or back-flow of gases during fluid infusion procedures while preventing the escape of liquid into the gas supply line 114.

During the fluid-air exchange, the infusion liquid may flow through the liquid supply line 116 and the flow-through member 258 of the lower body 202, while also flowing through the through channels 256 and into the cavity 248. The infusion liquid may occupy the volume between the ridges 252 and deform the filter 222 toward the first conduit 236 (e.g., push the filter 222 in an upwards direction towards the cover 238), until the filter 222 is pressed against the ridges 242 of the cover 238. When infusion gases from the gas supply line 114 reach the cavity 248 and contact the filter 222, the infusion gases cause the filter 222 to “diaphragm” down (e.g., push downwards) towards the ridges 252 of the base 250, thereby pushing infusion liquid through the channels in the ridges 252 to the through channels 256 and into the flow through member 258.

While the filter 222 is effective at preventing the flow of liquids past the hydrophobic membrane into the first cavity 260 (and toward the gas supply line 114), liquid flow into the filter 222 can cause the filter 222 to saturate, which hinders gas flow through the filter 222. The combination of the activation member 228 and the filter 222 provides a low surface tension region between the facing hydrophobic surfaces. Responsive to increased gas flow, the first cavity 260 pressurizes and deforms the filter 222 downward. This deformation causes increased surface area contact between the hydrophobic surfaces of the filter 222 and the activation member 228 (in some cases, the deformation initiates the contact therebetween), which results in a squeezing effect that forces liquid out, from the filter 222 and/or the activation member 228 near their circumferential edges, medially toward the second cavity 261 through the one or more openings (e.g., the central opening 230). Voids (also referred to as “air gaps” or “bubbles”) are formed behind the forced-out liquid and expand as more liquid is released from pores of the filter 222 and directed medially. Thus, the formation of the voids facilitates gas flow through the filter 222, e.g., during fluid-air exchange operations.

In some embodiments, a radiation stable membrane may be used (e.g., made of PVDF) with a silicone or PDMS ring. Radiation stable hydrophobic membranes may inhibit water from passing through them, however their surfaces (unlike PTFE membranes) may be susceptible to surface wetting under pressure. This wetting may inhibit air from passing through the membrane under low pressures (which may occur during air exchange). Therefore, in these embodiments, a silicone/PDMS ring is used such that when liquid is flowing, the liquid pressure pushes the silicone/PDMS ring up against the hydrophobic radiation stable membrane. This seals the pores of the radiation stable membrane, that are in contact with the silicone/PDMS ring, from the incoming water pressure, which in turn prevents or limits the amount of water able to wet or semi-penetrate the pores of the radiation stable membrane. Once the pressure is removed and air pressure is applied on the back side of the radiation stable membrane, the silicone/PDMS ring releases from the radiation stable membrane surface. This motion of release backed by air pressure allows a localized bubble to form and in turn allows for the silicone/PDMS ring to separate from the radiation stable membrane. This separation allows air flow through the radiation stable membrane.

While the activation member 228 is shown as ring-shaped with the central opening 230 being circle-shaped, other shapes and arrangements of shapes are also contemplated for the activation member 228. In one alternate example, the activation member 228 may be substantially circular (e.g., matching the shape of the filter 222) with a plurality of openings that are smaller than the central opening 230 depicted in FIG. 2C. Similarly, the opening(s) extending through the activation member 228 may have any suitable geometry, such as circles, slits, stars, and so forth, as well as combinations of different shapes. The opening(s) may be provided with any suitable arrangement, such as a regular arrangement of openings (e.g., in radial or rectangular coordinate may not be disposed circumferentially around the first axis A1, as well as an irregular arrangement of openings.

The choice of material for the activation member 228 may facilitate the assembly process of the valve assembly 200. In some embodiments, the activation member 228 is formed of a material with a lesser hardness than that of the hydrophobic filter 222. For example, the activation member 228 may be formed of a UHMWPE material, and the hydrophobic membrane of the filter 222 may be formed of ePTFE. In this way, the activation member 228 can shield the more sensitive hydrophobic membrane from particles or (incidental) contact that can occur during the assembly process, or in some cases, during operation. For example, the activation member 228 can largely prevent the lower surface of the filter 222 from sustaining micro-scratches and damage that hinder the performance of the filter 222. The activation member 228 can also protect the filter 222 from contaminants such as oils or residues. This improves the overall reliability of the valve assembly 200, as the hydrophobic membrane is shielded from holes, punctures, contaminants, etc. that may diminish the performance and/or the longevity of the filter 222.

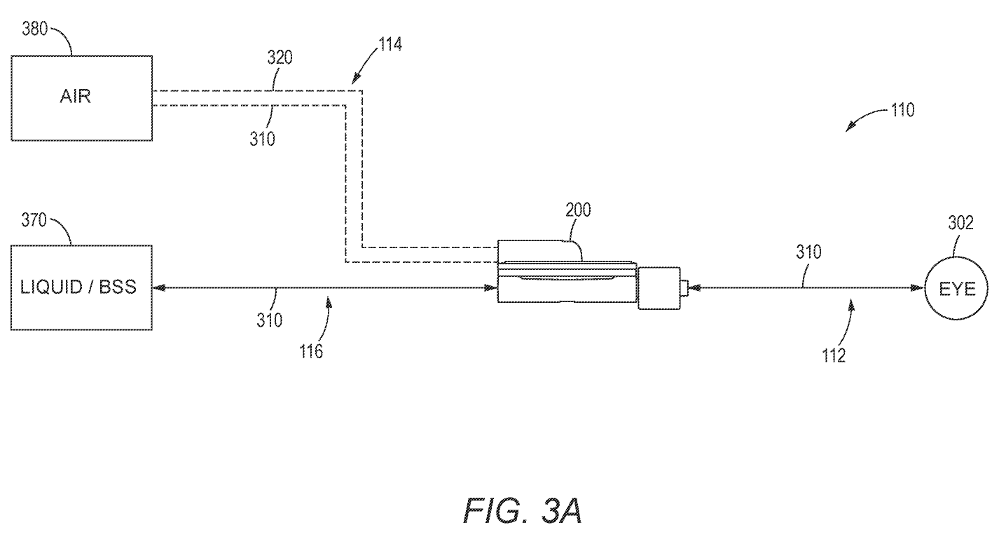

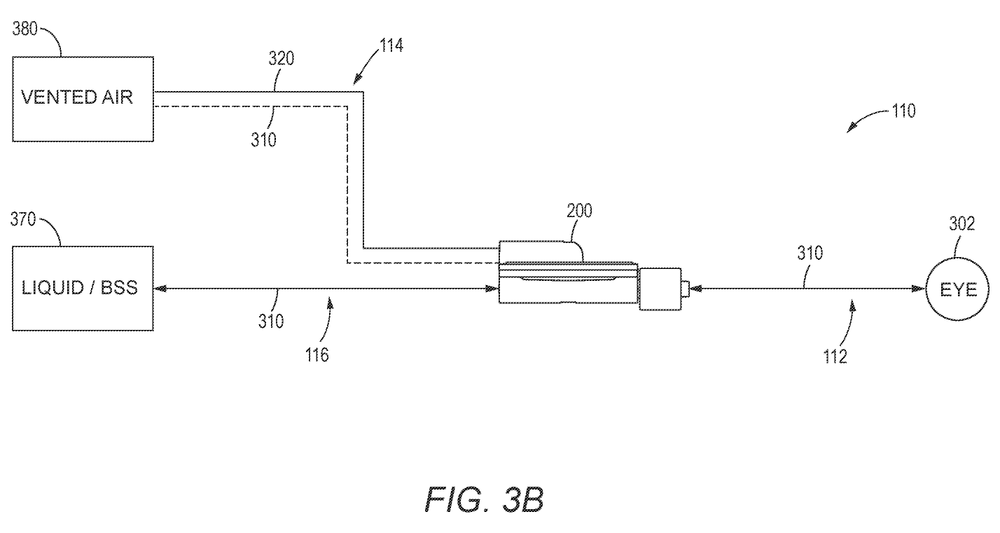

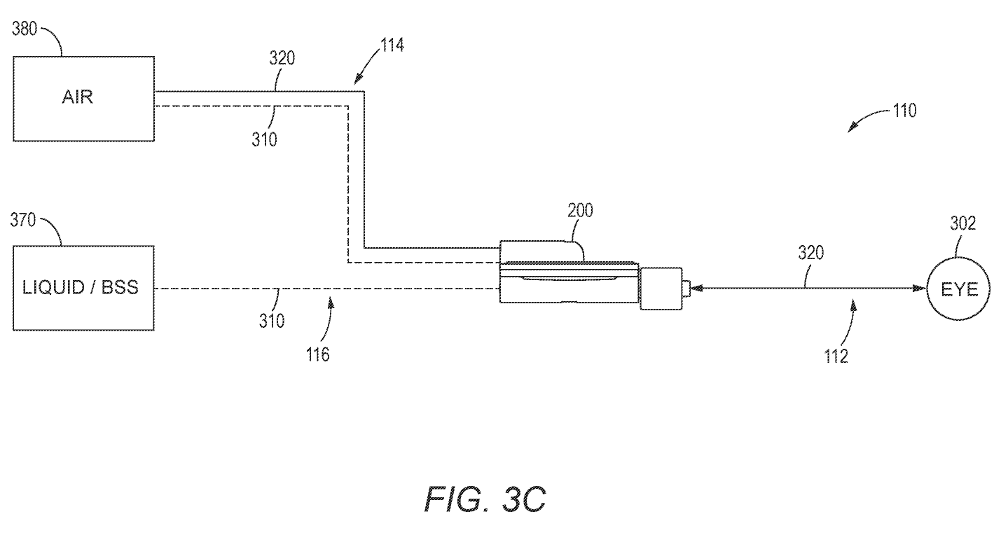

FIGS. 3A-3C schematically illustrate operational modes of the valve assembly 200 during fluid infusion procedures. In particular, FIGS. 3A-3C illustrate the flow of liquid solutions (e.g., BSS), represented by lines 310, and the flow of gases (e.g., air), represented by lines 320, through the fluid infusion system 110 having the valve assembly 200, as described above. Further, please note that unbroken lines (e.g., continuous lines) represent open or active flow, while broken lines (e.g., dashed lines) represent closed or no flow.

FIG. 3A depicts the fluid infusion system 110 during a first operation of liquid infusion, which may be selected and/or controlled by a user (e.g., a surgeon) via a surgical console, such as surgical console 100. As shown, infusion liquid (through the line 310) is controllably flowed between the liquid source 370 and eye 302 via liquid supply line 116, valve assembly 200, and infusion line 112, while air or gas flow through gas supply line 114 is stopped or shut off. To control a pressure within the fluid infusion system 110 and thus, the eye 302, the user may adjust the direction and flow rate of the liquid (through the line 310) to or from the liquid source 370 via the surgical console 100. The valve assembly 200 enables liquid (through the line 310) to flow between the liquid supply line 116 and the infusion line 112, while also preventing the liquid (through the line 310) from flowing into the gas supply line 114 and towards the gas source 380 due to the presence of the hydrophobic filter 222. Accordingly, the valve assembly 200 provides a passive means of preventing leakage of liquid (through the line 310) into gas supply line 114, which contrasts with conventional flow control valves that may allow the escape of at least some liquid (through the line 310) into the gas supply line 114 during use thereof.

FIG. 3B depicts the fluid infusion system 110 during a second operation of liquid infusion in which the pressure of air (through the line 320) within the gas supply line 114 is actively modulated while infusion liquid (through the line 310) is flowed between the liquid source 370 and eye 302. As described above, the pressure within the fluid infusion system 110 and the eye 302 is controlled by adjusting the direction and flow rate of the liquid (through the line 310) to or from the liquid source 370 via the surgical console 100. When left unchecked, pressure within the gas supply line 114 may inadvertently build up during infusion and cause air (through the line 320) to leak into the liquid (through the line 310) being injected into the eye 302, thereby negatively affecting the intraocular pressure thereof. Therefore, in certain embodiments, it may be desired to apply a vacuum pressure (e.g., negative pressure) to the gas supply line 114 to vent the gas supply line 114 and prevent the undesired escape of air (through the line 320) into the liquid (through the line 310) as bubbles. In certain embodiments, active venting of the gas supply line 114 may also be desired to purge the infusion liquid (through the line 310) of gases already trapped therein as the liquid (through the line 310) passes into the infusion line 112.

Since conventional flow control valves cannot prevent the leakage of liquid (through the line 310) into the gas supply line 114, venting of the gas supply line 114 with a conventional valve is extraordinarily difficult. In comparison, as a result of the hydrophobic filter 222, the valve assembly 200 facilitates active venting of the gas supply line 114 during infusion of liquid (through the line 310) into the eye 302, thus reducing or eliminating the possibility of unwanted gases being flowed into eye 302 and disrupting the intraocular pressure therein.

FIG. 3B is further representative of the fluid infusion system 110 during an infusion fluid back-flow operation. Back-flow of infusion fluids may be necessitated when the eye 302 is injected, via a separate cannula or injection device, with a retinal tamponade (or other fluid treatment) such as intraocular air/gas, silicone oil, or perfluoron. As a result, infusion fluids previously flowed through the infusion line 112 may need to be back-flowed. Because conventional flow control valves cannot backflow or purge gases into the gas supply line 114 without leakage of infusion liquid, only a limited volume of infusion fluids can be back-flowed without risking the chance of liquid leakage into the gas supply line 114 or gas leakage into the liquid supply line 116. In contrast, the hydrophobic filter 222 of the valve assembly 200 in FIG. 3B enables backflow of gases into the gas supply line 114 without leakage of infusion liquids, thus allowing a greater volume of the infusion fluids to be back-flowed into their respective supply lines and further enabling a greater volume of treatment fluids to be injected into the eye 302.

FIG. 3C depicts the fluid infusion system 110 during a third operation of liquid infusion. The operational mode depicted in FIG. 3C may be performed, for example, during a fluid-air exchange to help push out subretinal fluid from the intraocular space of the eye 302. As shown, air (through the line 320) is flowed from the gas source 380 to the eye 302, while liquid flow through the liquid supply line 116 is shut off to prevent escape of liquid (through the line 310) into the infused air (through the line 320). Accordingly, the pressure within the fluid infusion system 110 and the eye 302 is controlled by adjusting the direction and flow rate of the air (through the line 320) to or from the gas source 380 via the surgical console 100.

In summary, embodiments of the present disclosure include structures and mechanisms for improved intraocular pressure maintenance during ophthalmic procedures, and in particular, improved fluid control valves for intraocular fluid infusion. The valve assemblies described above include an apparatus comprising a valve body comprising a first conduit defining a first port, and a second conduit defining a second port and a third port. A cavity is defined in the valve body between the first conduit and the second conduit. The cavity is fluidly coupled with the first conduit via a first channel having a diameter smaller than that of the first conduit, and with the second conduit via at least one second channel. The apparatus further comprises a hydrophobic filter attached to the valve body and extending substantially across the cavity. The apparatus further comprises an activation member disposed in the cavity and defining one or more openings extending from a first side of the activation member contacting the hydrophobic filter, to an opposing second side facing the second conduit.

The many features and advantages of disclosure are apparent from detailed specification, and, thus, it is intended by appended claims to cover all such features and advantages of disclosure which fall within scope of disclosure. Further, since numerous modifications and variations will readily occur to those skilled in art, it is not desired to limit disclosure to exact construction and operation illustrated and described, and, accordingly, all suitable modifications and equivalents may be resorted to that fall within scope of disclosure.

### EXAMPLE EMBODIMENTS

Embodiment 1. A fluid infusion system for ophthalmic procedures, the fluid infusion system comprising: a surgical console comprising: a first fluid line coupled to a gas fluid source; and a second fluid line coupled to a liquid fluid source. The fluid infusion system further comprises a valve assembly fluidly coupled to the first fluid line and the second fluid line. The valve assembly comprises: a valve body comprising: a first conduit defining a first port; and a second conduit defining a second port and a third port, wherein a cavity is defined in the valve body between the first conduit and the second conduit, the cavity fluidly coupled with the first conduit via a first channel having a diameter smaller than that of the first conduit, and with the second conduit via at least one second channel. The valve assembly further comprises a hydrophobic filter attached to the valve body and extending substantially across the cavity; and an activation member disposed in the cavity and defining one or more openings extending from a first side of the activation member contacting the hydrophobic filter, to an opposing second side facing the second conduit.

Embodiment 2. The fluid infusion system of Embodiment 1, wherein the hydrophobic filter comprises: a hydrophobic membrane that contacts a hydrophobic surface at the first side of the activation member, the hydrophobic membrane configured to: prevent liquid flow through the hydrophobic filter toward the first conduit; and allow bidirectional gas flow through the hydrophobic filter.

Embodiment 3. The fluid infusion system of Embodiment 2, wherein the hydrophobic surface comprises a second hydrophobic membrane disposed at the first side of the activation member.

Embodiment 4. The fluid infusion system of Embodiment 1, wherein the hydrophobic filter is configured to: deform toward the first conduit responsive to liquid flow from the second conduit into the cavity; and thereafter, deform toward the second conduit responsive to gas flow from the first conduit into the cavity.

Embodiment 5. The fluid infusion system of Embodiment 4, wherein the valve body further defines: one or more surfaces that partially define the cavity, the one or more surfaces comprising one or more semi-annular or annular ridges defining one or more channels. The hydrophobic filter is further configured to, when deforming toward the first conduit or toward the second conduit, contact the one or more semi-annular or annular ridges.

Embodiment 6. The fluid infusion system of Embodiment 1, wherein the first channel, the cavity, and the at least one second channel are coaxially disposed about a first axis.

Embodiment 7. The fluid infusion system of Embodiment 6, wherein the at least one second channel comprises two or more second channels disposed circumferentially about the first axis.

Embodiment 8. The fluid infusion system of Embodiment 6, wherein the one or more openings comprises a central opening disposed about the first axis.

Embodiment 9. The fluid infusion system of Embodiment 1, wherein the valve body comprises: a cover member defining the first conduit and the first channel; and a base member defining the second conduit and the at least one second channel.

Embodiment 10. The fluid infusion system of Embodiment 1, wherein the activation member is attached to the hydrophobic filter.

Embodiment 11. The fluid infusion system of Embodiment 1, wherein the activation member is formed of a same material as the hydrophobic filter.

Embodiment 12. The fluid infusion system of Embodiment 1, wherein the activation member is formed of a material with a lesser hardness than that of the hydrophobic filter.

Embodiment 13. The fluid infusion system of Embodiment 1, wherein the one or more openings comprises a plurality of openings.

What is claimed is:

1. An apparatus to perform fluid infusion during surgical procedures, the apparatus comprising:
a valve body comprising:
a first conduit defining a first port; and
a second conduit defining a second port and a third port, wherein a cavity is defined in the valve body between the first conduit and the second conduit, the cavity fluidly coupled with the first conduit via a first channel having a diameter smaller than that of the first conduit, and with the second conduit via at least one second channel;
a hydrophobic filter attached to the valve body and extending substantially across the cavity; and
an activation member disposed in the cavity and defining one or more openings extending from a first side of the activation member contacting the hydrophobic filter, to an opposing second side facing the second conduit.

2. The apparatus of claim 1, wherein the hydrophobic filter comprises:
a hydrophobic membrane that contacts a hydrophobic surface at the first side of the activation member,
the hydrophobic membrane configured to:
prevent liquid flow through the hydrophobic filter toward the first conduit; and
allow bidirectional gas flow through the hydrophobic filter.

3. The apparatus of claim 2, wherein the hydrophobic surface comprises a second hydrophobic membrane disposed at the first side of the activation member.

4. The apparatus of claim 1, wherein the hydrophobic filter is configured to:
deform toward the first conduit responsive to liquid flow from the second conduit into the cavity; and
thereafter, deform toward the second conduit responsive to gas flow from the first conduit into the cavity.

5. The apparatus of claim 4, wherein the valve body further defines:
one or more surfaces that partially define the cavity, the one or more surfaces comprising one or more semi-annular or annular ridges defining one or more channels,
wherein the hydrophobic filter is further configured to, when deforming toward the first conduit or toward the second conduit, contact the one or more semi-annular or annular ridges.

6. The apparatus of claim 1, wherein the first channel, the cavity, and the at least one second channel are coaxially disposed about a first axis.

7. The apparatus of claim 6, wherein the at least one second channel comprises two or more second channels disposed circumferentially about the first axis.

8. The apparatus of claim 6, wherein the one or more openings comprises a central opening disposed about the first axis.

9. The apparatus of claim 1, wherein the valve body comprises:
a cover member defining the first conduit and the first channel; and
a base member defining the second conduit and the at least one second channel.

10. The apparatus of claim 9, wherein the hydrophobic filter is coupled to the cover member and then the cover member is coupled to the base member.

11. The apparatus of claim 1, wherein the activation member is attached to the hydrophobic filter.

12. The apparatus of claim 1, wherein the activation member is formed of a same material as the hydrophobic filter.

13. The apparatus of claim 1, wherein the activation member is formed of a material with a lesser hardness than that of the hydrophobic filter.

14. The apparatus of claim 1, wherein the one or more openings comprises a plurality of openings.

15. The apparatus of claim 1, wherein the hydrophobic filter is a membrane made of at least one of polytetrafluoroethylene (PTFE), expanded PTFE (ePTFE), polycarbonate track etch (PCTE), polyester, nylon, cellulose, cellulose nitrate (CN), cellulose acetate (CA), polyethersulfone (PES), glass fibers, or acrylic copolymers.

16. The apparatus of claim 1, wherein the hydrophobic filter is a radiation stable polyvinylidene difluoride (PVDF) membrane and the activation member is a silicone or polydimethylsiloxane (PDMS) ring.

17. A fluid infusion system for surgical procedures, the fluid infusion system comprising:
a surgical console comprising:
a first fluid line coupled to a gas fluid source; and
a second fluid line coupled to a liquid fluid source; and
a valve assembly fluidly coupled to the first fluid line and the second fluid line, the valve assembly comprising:
a valve body comprising:
a first conduit defining a first port; and
a second conduit defining a second port and a third port, wherein a cavity is defined in the valve body between the first conduit and the second conduit, the cavity fluidly coupled with the first conduit via a first channel having a diameter smaller than that of the first conduit, and with the second conduit via at least one second channel;
a hydrophobic filter attached to the valve body and extending substantially across the cavity; and
an activation member disposed in the cavity and defining one or more openings extending from a first side of the activation member contacting the hydrophobic filter, to an opposing second side facing the second conduit.

18. The fluid infusion system of claim 17, wherein the hydrophobic filter comprises:
a hydrophobic membrane that contacts a hydrophobic surface at the first side of the activation member,
the hydrophobic membrane configured to:
prevent liquid flow through the hydrophobic filter toward the first conduit; and
allow bidirectional gas flow through the hydrophobic filter.
